(19) United States
(12) Patent Application Publication
(10) Pub. No.: US 2016/0122795 A1
(43) Pub. Date: May 5, 2016

(54) MICROORGANISM CULTURING MATERIAL AND METHOD FOR DETECTING MICROORGANISMS

(71) Applicant: JNC CORPORATION, Tokyo (JP)

(72) Inventor: Hajime TERAMURA, Kanagawa (JP)

(21) Appl. No.: 14/925,987

(22) Filed: Oct. 29, 2015

(30) Foreign Application Priority Data
Oct. 29, 2014 (JP) 2014-220008

(51) Int. Cl. C12Q 1/04

(52) U.S. Cl. CPC C12Q 1/045

(57) ABSTRACT

A sheet-form microorganism culturing material including a water-soluble polymer compound layer and a porous matrix layer in which both high moisture retention capability and colony color formation capability are satisfied.

The microorganism culturing material includes a first layer containing a porous material, and a second layer adjacent to the first layer and containing a gelling agent, wherein the microorganism culturing material contains polyvinyl alcohol and methylcellulose or guar gum as the gelling agent.

### CROSS-REFERENCE TO RELATED APPLICATIONS

This application claims the priority benefit of Japan application serial no. 2014-220008, filed on Oct. 29, 2014. The entirety of the above-mentioned patent application is hereby incorporated by reference herein and made a part of this specification.

### TECHNICAL FIELD

The present invention relates to a material and a culture medium for culturing microorganisms, and a method for detecting microorganisms using the same.

### BACKGROUND ART

As a simple culture medium for counting the number of microorganisms in food testing or the like, media of various types have been developed, and specific examples include a stamp type, a filter type, a film type, a test paper type and a sheet type.

Above all, specific examples of media of the sheet type include a sheet-form culturing material containing a water-soluble polymer compound layer containing polyvinyl alcohol, and a porous matrix layer, and such a material is known to be applicable to various applications such as food testing (Patent literature Nos. 1 to 2).

The sheet-form culturing materials in Patent literature Nos. 1 to 2 are in a dry state before use, and if a liquid sample (specimen) is added to the porous matrix layer during use, the liquid sample is temporarily held in the whole porous matrix layer due to a capillary action, and then moisture in the liquid sample allows dissolution and swelling of polyvinyl alcohol in the water-soluble polymer compound layer in contact with the porous matrix layer to form a culture medium integrated with the porous matrix layer. In such a culture medium, the microorganisms in the liquid sample grow to form colonies on a surface of the porous matrix layer. Therefore, colored colonies can be easily detected by incorporating a color former or the like into the culture medium.

Thus, the sheet-form culturing material including the water-soluble polymer compound layer and the porous matrix layer can simplify a series of operations of preparation of the culture medium, inoculation of the specimen and detection of the colonies to realize efficient detection of the microorganisms.

### CITATION LIST

### Patent Literature

Patent literature No. 1: WO 97/24432 A.

Patent literature No. 2: WO 01/44437 A.

### SUMMARY OF INVENTION

### Technical Problem

However, polyvinyl alcohol contained in sheet-form culturing materials in Patent literature Nos. 1 to 2 has high viscosity during dissolution and swelling due to moisture contained therein, and therefore flowability of free water in a gelated culture medium is reduced. Then, if an amount of a color former or the like to be incorporated into the culture medium is adjusted to a degree of an amount of use in a general agar culture medium, contact between the microorganisms and the color former becomes poor, and therefore color formation of the colonies becomes weak and detection thereof becomes difficult in several cases.

An increase in the amount of color former or the like in order to solve such a problem is in circumstances of being desirably avoided from a viewpoint of suppressing cost of the culturing material to be used in a large amount and in a disposal manner in routine testing because the color former or the like is expensive.

Moreover, minimization of a content of polyvinyl alcohol or reduction of a degree of polymerization thereof can also be considered in order to reduce the viscosity of polyvinyl alcohol during dissolution and swelling thereof. However, while the colony color formation can be enhanced, moisture retention capability of the culture medium is weakened. Therefore, the colonies are diffused in the culture medium to cause difficulty in detection thereof, or the culture medium flows out from a matrix to cause a risk of contamination due to the microorganisms.

In view of such a situation, the invention is contemplated for providing a microorganism culturing material in which colony color formation capability is enhanced without reducing the moisture retention capability in the water-soluble polymer compound layer in the sheet-form culturing material including the water-soluble polymer compound layer and the porous matrix layer.

### Solution to Problem

The present inventors have diligently continued to conduct study in order to solve the problems as described above. As a result, the present inventors have founded that both high moisture retention capability and colony color formation capability can be satisfied by combining methylcellulose or guar gum with polyvinyl alcohol, as a water-soluble polymer compound to be incorporated into a water-soluble polymer compound layer, and have completed the invention.

More specifically, the invention is as described below.

Item 1. A microorganism culturing material, including a first layer containing a porous material, and a second layer adjacent to the first layer and containing gelling agent, wherein the gelling agent contains polyvinyl alcohol, and methylcellulose or guar gum.

Item 2. The microorganism culturing material according to item 1, wherein a weight ratio of polyvinyl alcohol to methylcellulose is 90:10 to 50:50.

Item 3. The microorganism culturing material according to item 1, wherein a weight ratio of polyvinyl alcohol to guar gum is 90:5 to 90:25.

Item 4. The microorganism culturing material according to any one of items 1 to 3, wherein a total amount of gelling agent contained in the second layer is 50 to 200 g/m².

Item 5. The microorganism culturing material according to any one of items 1 to 4, wherein the polyvinyl alcohol has a weight average molecular weight of 5,000 to 200,000, and a degree of saponification of 75 to 99%.

Item 6. The microorganism culturing material according to any one of items 1 to 5, further including a third layer adjacent to the second layer on a side opposite to the first layer.

Item 7. A microorganism culture medium, containing the microorganism culturing material according to any one of items 1 to 6, at least one kind of color former or fluorescence agent, and at least one kind of nutritional ingredient.

Item 8. A method for detecting microorganisms, including a step of inoculating a specimen to the microorganism culture medium according to item 7, a step of culturing the microorganisms contained in the specimen, and a step of detecting colonies of the microorganisms.

### Advantageous Effects of Invention

The invention provides an inexpensive microorganism culturing material that can simply and efficiently detect microorganisms in a specimen as clear colored colonies.

### BRIEF DESCRIPTION OF DRAWINGS

FIG. 1 are photographs showing colored colonies on culture media in Example 1.

FIG. 2 are photographs showing colored colonies on culture media in Example 2.

FIG. 3 are photographs showing colored colonies on culture media in Example 3.

### DESCRIPTION OF EMBODIMENTS

A microorganism culturing material of the invention includes a first layer containing a porous material, and a second layer adjacent to the first layer and containing a gelling agent.

The first layer is a layer that plays a role of a matrix in the microorganism culturing material. More specifically, the layer is a support of the microorganism culturing material, and a base for holding and integrating the gelling agent dissolved and swollen during use and contained in the second layer to be infiltrated and dispersed thereinto.

The second layer is a layer that plays a role of retaining moisture in the culture medium in the microorganism culturing material, and a role of serving as the culture medium during use to allow the microorganisms to grow and to form the colonies.

Specific examples of the porous material contained in the first layer include a knitted or woven fabric or a nonwoven fabric formed of synthetic fibers, semisynthetic fibers, natural fibers and inorganic fibers, a porous film and sponge. Porous ceramics may be used. Specific examples of the synthetic fibers include nylon, polyacrylonitrile, polyvinyl alcohol, an ethylene-vinyl acetate copolymer, polyester that may be subjected to hydrophilic treatment, a polyolefin that may be subjected to hydrophilic treatment and polyurethane. Specific examples of the semisynthetic fibers include rayon. As the natural fibers, wool, silk, cotton, cellulose, pulp or the like is preferred.

In particular, a kitted or woven fabric, a nonwoven fabric or the like in which adjustment of unit weight or air permeability is easy is preferred, and a nylon meltblown nonwoven fabric prepared by a meltblown manufacturing method according to which fine fibers can be comparatively easily obtained, or an ultrafine fiber nonwoven fabric manufactured from splittable fibers is further preferred.

Moreover, the unit weight of the porous material is preferably approximately 50 to approximately 90 g/m², and further preferably approximately 55 to approximately 80 g/m². If the unit weight is in the range described above, moisture retention capability in the microorganism culturing material can be sufficiently easily secured, no liquid sample (specimen) overflows from the first layer, and the first layer and the second layer can be sufficiently integrated. Therefore, colonies each having a good shape and size to be easily observable are easily formed. Moreover, the microorganisms are pushed up onto a surface of the first layer, and therefore the microorganisms grow only on the surface of the first layer to facilitate detection of the colonies, and growth of the microorganisms becomes easily sufficient.

Moreover, the air permeability of the porous material is preferably approximately 7 to approximately 24 cm/sec (70 to 240 L/(m²·sec)), further preferably approximately 8 to approximately 20 cm/sec, and still further preferably approximately 10 to approximately 18 cm/sec. If the air permeability is in the range described above, moisture is uniformly easily permeated into the second layer upon adding the liquid sample (specimen) thereto, and the microorganisms are uniformly easily cultured. Moreover, fixing properties of the gelling agent contained in the second layer upon dissolution and swelling thereof are sufficiently easily secured, and therefore the colonies each having the good shape and size to be easily observable are easily formed. In addition, the air permeability is measured by Frazier Type Method specified in JIS L1096 8.26.

The gelling agent contained in the second layer includes a combination of polyvinyl alcohol and methylcellulose or a combination of polyvinyl alcohol and guar gum.

A weight ratio of polyvinyl alcohol to methylcellulose is preferably approximately 90:10 to approximately 50:50 because color formation of the colonies and the size thereof become satisfactory, and further preferably approximately 90:10 to approximately 75:25 also from viewpoints of solubility during preparation of the second layer and capability of uniformly applying the mixture onto the first layer.

A weight ratio of polyvinyl alcohol to guar gum is preferably approximately 90:5 to approximately 90:25 because the color formation of the colonies and the size thereof become satisfactory, and further preferably approximately 90:5 to approximately 90:20 also from viewpoints of solubility during preparation of the second layer and capability of uniformly applying the mixture onto the first layer.

Polyvinyl alcohol preferably has a weight average molecular weight of approximately 5,000 to approximately 200,000 and a degree of saponification of approximately 75 to approximately 99%.

Methylcellulose preferably has a weight average molecular weight of approximately 100,000 to approximately 1,000,000.

Guar gum preferably has a weight average molecular weight of approximately 100,000 to approximately 500,000.

When the second layer contains moisture, the gelling agent has viscosity capable of preferably satisfying both the moisture retention capability and the color formation capability by each material being in the range described above.

A total amount of the gelling agent contained in the second layer according to the invention is preferably approximately 50 to approximately 200 g/m² in terms of an application amount per unit area of the first layer. The total amount is within such a range. Thus, the microorganism culturing material can be easily prepared, and simultaneously the microorganisms easily grow, and counting of the colonies on the culture medium is facilitated.

Moreover, the gelling agent may contain any other gelling agent in addition to the combination of polyvinyl alcohol and methylcellulose or the combination of polyvinyl alcohol and guar gum. However, an amount of preferably approximately 80% by weight or more of the total amount of the gelling agent, further preferably approximately 90% by weight or more, and still further preferably approximately 95% by weight of more is in the combination of polyvinyl alcohol and methylcellulose or the combination of polyvinyl alcohol and guar gum.

Specific examples of any other gelling agent include a cellulose derivative such as carboxymethylcellulose and hydroxyalkylcellulose; starch and a derivative thereof; polysaccharide such as hyaluronic acid and xanthan gum; an acrylic acid derivative such as polyacrylic acid, polyacrylate and an acrylic acid-vinyl alcohol copolymer; polyether such as polyethylene glycol and polypropylene glycol; and protein such as collagen.

A mixture of the gelling agent contained in the second layer according to the invention exhibits preferably approximately 10 mPa·s or more, and further preferably approximately 15 to approximately 80 mPa·s in terms of the viscosity of a 4 wt % aqueous solution as measured at 20° C. by using an Ostwald viscometer. The mixture has such viscosity. Thus, the microorganisms neither invade into the culture medium nor move on a surface of the culture medium, and the microorganisms form the colonies mainly on the surface of the culture medium to facilitate detection and counting of the colonies.

The microorganism culturing material of the invention may further include a third layer. The third layer is preferably adjacent to a surface of the second layer on a side opposite to a surface to which the first layer is adjacent.

The third layer plays a role of reinforcing the microorganism culturing material or protecting the second layer to facilitate transportation, storage and handling thereof.

As a material to be used as the third layer, a water-insoluble synthetic resin is preferred, for example, and specific examples preferably include polyester, nylon and a polyolefin. A shape thereof is preferably a film-form or a sheet-form, and a thickness thereof is preferably approximately 0.03 to approximately 2 millimeters, and further preferably approximately 0.04 to approximately 0.2 millimeters.

If at least one kind of color former or fluorescent agent and at least one kind of nutritional ingredient are further incorporated into the microorganism culturing material according to the invention, the resulting composition serves as a microorganism culture medium of the invention.

The color former or fluorescent agent is applied for allowing formation of color or generation of fluorescence of the colonies of the microorganisms grown on the culture medium to facilitate viewing of the colonies and to facilitate detection of the colonies and counting thereof in a method for detecting the microorganisms described later.

The color former or fluorescent agent preferably serves as a substrate of an enzyme, and more specifically, can liberate a colored or fluorescent chromogenic compound by an enzyme reaction. For example, when the microorganism to be detected are a coli group, specific examples include 5-bromo-4-chloro-3-indolyl-β-D-galactopyranoside (X-GAL) serving as a substrate of β-galactosidase, and when the microorganisms are Escherichia coli, specific examples preferably include 5-bromo-4-chloro-3-indoxyl-β-D-glucuronic acid or also a salt thereof. Moreover, when the microorganisms are Staphylococcus aureus, specific examples preferably include 5-bromo-4-chloro-3-indoxyl-phosphoric acid (X-phos) or a salt thereof.

The color former or fluorescence agent is preferably adjusted to have a content of approximately 0.01 to approximately 1.0 g per m² of the microorganism culturing material. In the microorganism culturing material of the invention, flowability of free water in the culture medium is secured without causing excessively high viscosity upon dissolving and swelling of the gelling agent in the second layer. Therefore, good color formation of the colonies can be secured even without increasing an amount of the color former or fluorescent agent.

The nutritional ingredient is applied for growing the microorganisms in the culture medium. Specific examples include peptone, soy peptone, a yeast extract, an animal meat extract, a fish meat extract, glucose, sucrose and lactose.

Moreover, a selective substance for selectively growing specific microorganisms in the culture medium may be incorporated thereinto. Specific examples of the selective substance include an antibiotic, a synthetic antimicrobial agent, a dye, a surfactant and an inorganic salt. Specific examples of the antibiotic include methicillin, cefmetazole, cefixime, ceftazidime, cefsulodin, bacitracin, polymyxin B, rifampicin, novobiocin, colistin, lincomycin, chloramphenicol, tetracycline and streptomycin. Specific examples of the synthetic antimicrobial agent include a sulfa drug, nalidixic acid and olaquindox. Specific examples of the dye include crystal violet, brilliant green, malachite green and methylene blue, each having bacteriostatic or bactericidal action. Specific examples of the surfactant include Tergitol 7, dodecyl sulfate and lauryl sulfate. Specific examples of the inorganic salts include selenite, tellurite, sulfite, sodium nitride, lithium chloride, oxalate and sodium chloride. In addition thereto, taurocholate, glycine, bile powder, bile salt, deoxycholate or the like may be used.

In addition thereto, an ingredient ordinarily used in the microorganism culture medium, such as a pH adjuster, may be arbitrarily incorporated into the culture medium of the invention.

Moreover, the microorganism culture medium of the invention may contain moisture. However, from viewpoints of storage stability and handling properties, a content thereof is preferably ordinarily approximately 50% by weight or less, and further preferably approximately 30% by weight or less in a state before use. Moreover, during use (during growing the microorganisms), the content is preferably adjusted to approximately 350 to approximately 650 mL per m² of the microorganism culturing material. More specifically, the microorganism culturing material of the invention is in a dry state before use, but during use, the liquid sample (specimen) is added thereto to allow dissolution or swelling of the gelling agent with the moisture to be formed into a state in which the microorganisms can grow.

The microorganism culturing material and the microorganism culture medium according to the invention are not particularly limited, but can be prepared by procedures described below, for example.

A constituent containing the gelling agent in the second layer is mixed to water and dissolved thereinto, and the resulting mixture is applied onto an insoluble film or the like, and then dried to form the second layer, and the second layer is laminated onto the porous material constituting the first layer. Alternatively, a solution of the gelling agent may be applied onto the second layer, and then dried. Then, a film or the like constituting the third layer is laminated onto the surface of the second layer on a side opposite to the surface to which the first layer is adjacent. Into the thus prepared sheet-form microorganism culturing material, the color former or fluorescent agent, and the nutritional ingredient are impregnated, deposited or adsorbed, and dried, and the resulting material is served as the microorganism culture medium. Alternatively, the color former or fluorescent agent, and the nutritional ingredient are also mixed to water and dissolved thereinto together with the constituent of the second layer, and then the microorganism culture medium may be prepared through coating, drying and so forth as described above.

The microorganism culture medium of the invention can be preferably utilized in the method for detecting the microorganisms in the specimen. The method includes a step of inoculating the specimen to the microorganism culture medium, a step of culturing the microorganisms contained in the specimen and a step of detecting the colonies of the microorganisms according to the invention. In the microorganism culture medium of the invention, the colonies grow large and the color formation becomes clear, and therefore according to the detection method of the invention, detection and counting of the microorganisms can be easily performed.

In the step of inoculating the specimen to the microorganism culture medium, the specimen may be inoculated from any sides of the first layer and the second layer, but is preferably inoculated from the side of the first layer. In the above case, the specimen (ordinarily liquid sample) is diffused into the porous material in the first layer in a horizontal direction and a vertical direction, and reaches the second layer, in which moisture in the specimen dissolves the gelling agent in the second layer, and the resulting solution is infiltrated into the porous material in the first layer, and thus the first layer and the second layer are integrated. Moreover, the nutritional ingredient or the like in the culture medium is also dissolved out, and therefore growth of the microorganisms starts. The solution of the gelling agent has high viscosity. Therefore, no microorganisms enter into an inside of the second layer, and the microorganisms are pushed up onto the surface of the culture medium (on the first layer on a side opposite to the surface adjacent to the second layer) to form the colonies thereon.

In the step of culturing the microorganisms, a culture temperature and a culture time suitable for the microorganisms to be detected can be appropriately selected.

Detection of the colonies formed thereon can be easily performed because the colonies are colored or fluoresced by the color former, fluorescent agent or the like.

Specific examples of the specimen to be applied to the detection method according to the invention include perishable foods such as meat, fish and shellfish, a clinical specimen such as feces, sea water and a wiping specimen in a cooking place, hospital or the like. Moreover, a culture fluid obtained by preculturing the specimens in Tryptic Soy Broth and Preston Broth or the like, and a culture fluid obtained by further culturing the culture fluid in a culture medium for proliferating microbial cells can also be used as the specimens.

In addition, the specimens are not particularly limited, but ordinarily are provided for testing as the liquid sample in a state of being dissolved or suspended into water, a liquid or the like in the culture medium.

Moreover, specific examples of the microorganisms to be grown in the microorganism culture medium or to be detected by the method for detecting the microorganisms according to the invention include Escherichia coli and a coliform bacteria, Staphylococcus species, Vibrio species, Enterococcus species and fungi, but are not particularly limited thereto.

It will be apparent to those skilled in the art that various modifications and variations can be made in the invention and specific examples provided herein without departing from the spirit or scope of the invention. Thus, it is intended that the invention covers the modifications and variations of this invention that come within the scope of any claims and their equivalents.

The following examples are for illustrative purposes only and are not intended, nor should they be interpreted to, limit the scope of the invention.

### EXAMPLES

The invention will be described in greater detail by way of Examples. The invention is not limited by the Examples.

### Reference Example

A base culture medium for Staphylococcus aureus was prepared at a formulation shown in Table 1. The base culture medium and 1 liter of each gelling agent in a use amount as shown in Table 2 were added to 1 liter of purified water, and the resulting mixture was warmed at 95° C. for 1 minute and dissolved with each other. Then, a total amount of the resulting solution was applied onto a polyester film having a thickness of 20 micrometers and 1 m×1 m, and resulting film was dried at 65° C. A product peeled from the polyester film was laminated onto a nylon meltblown nonwoven fabric (90 g/m²). Then, polyester films each having a thickness of 100 micrometers and 70 mm×80 mm were adhered onto a side opposite to the nonwoven fabric, and taken as sheet-form simple culture media (0-1) to (0-4).

TABLE 1
Table 1
Formation of base medium for Staphylococcus aureus (g/L)

| | |
|---|---|
| Peptone | 7.5 |
| Meat extract | 2.5 |
| Yeast extract | 1.5 |
| Mannitol | 5 |
| Lithium chloride | 2.5 |
| X-phos•2Na | 0.25 |
| Potassium tellurite | 0.005 |
| Sodium carbonate | 0.3 |

pH 7.4 ± 0.2

TABLE 2
Table 2

| | No. | | | |
|---|---|---|---|---|
| | 0-1 | 0-2 | 0-3 | 0-4 |
| Polyvinyl alcohol (g/L) | 120 | 0 | 0 | 0 |
| Carboxymethylcellulose | 0 | 120 | 0 | 0 |
| ammonium salt (g/L) | | | | |
| Carboxymethylcellulose | 0 | 0 | 120 | 0 |
| sodium salt (g/L) | | | | |
| Methylcellulose (g/L) | 0 | 0 | 0 | 120 |
| Aspect during preparation | Clearly dissolved | Not | Clearly | Separated |
| | | uniformly | dissolved, | after being |
| | | dissolved, | brittle after | heated and |
| | | high | drying, No | dissolved, |
| | | viscosity, | peeling | and |
| | | difficult in | | insoluble |
| | | dispensing | | matters |
| | | | | appeared |
| Size of colonies | Average | Small | Small | Large |
| Color formation of colonies | Somewhat | Somewhat | Somewhat | Clear |
| | lighter than | lighter than | lighter than | |
| | general | general | general | |
| | agar | agar | agar | |
| | medium | medium | medium | |

Staphylococcus aureus (NBRC 14462) was cultured for 24 hours on a Tryptic Soy Agar (made by Nissui Pharmaceutical Co., Ltd.), and then the resulting bacteria were suspended into sterile physiological saline using a sterile cotton swab so as to correspond to McFarland nephelometry No. 1 (approximately 3.0×10⁸ CFU/mL), and taken as a bacteria stock solution. Then, the bacteria stock solution was repeatedly subjected to 10-fold serial dilution to 10⁻⁷ by using sterile physiological saline, and then a 10⁻⁷ bacterial diluent was applied to each simple culture medium for testing in an amount of 500 mL/m² for each. The resulting simple culture media were cultured at 35° C. for 24 hours, and then a size and color formation of colonies were confirmed.

As shown in Table 2, all simple culture media were able to be prepared, but dried culture media other than simple culture medium (0-1) in which polyvinyl alcohol was used were brittle or no complete dissolution was caused, and therefore were hard to handle in producing a large amount, which had a problem. In simple culture media (0-2) and (0-3) in which two kinds of carboxymethylcellulose were used, colonies were found to be small. Moreover, in simple culture medium (0-4) in which methylcellulose was used, better color formation of the colonies was found in comparison with the medium in which polyvinyl alcohol was used.

### Example 1

A base culture medium for Staphylococcus aureus in a manner similar to the medium in Reference Example, and 1 liter of each gelling agent in a use amount as described in Table 3 were added to 1 liter of purified water, and the resulting mixture was warmed at 95° C. for 1 minute and dissolved with each other. Then, a total amount of the resulting solution was applied onto a polyester film having a thickness of 20 micrometers and 1 m×1 m, and resulting film was dried at 65° C. A product peeled from the polyester film was laminated onto a nylon meltblown nonwoven fabric (90 g/m²). Then, polyester films each having a thickness of 100 micrometers and 70 mm×80 mm were adhered onto a side opposite to the nonwoven fabric, and taken as sheet-form simple culture media (1-1) to (1-5).

In a manner similar to Reference Example, a 10⁻⁷ bacterial diluent of the bacteria solution of Staphylococcus aureus corresponding to McFarland nephelometry No. 1 was applied to each simple culture medium for testing in an amount of 500 mL/m² for each. The resulting simple culture media were cultured at 35° C. for 24 hours, and then a size and color formation of colonies were confirmed.

TABLE 3
Table 3

| | No. | | | | |
|---|---|---|---|---|---|
| | 1-1 | 1-2 | 1-3 | 1-4 | 1-5 |
| Polyvinyl alcohol | 120 | 114 | 108 | 90 | 60 |
| (g/L) | | | | | |
| Methylcellulose (g/L) | 0 | 6 | 12 | 30 | 60 |
| Polyvinyl | 100:0 | 95:5 | 90:10 | 75:25 | 50:50 |
| alcohol:Methylcellulose | | | | | |
| (weight ratio) | | | | | |
| Aspect during | Clearly | Clearly | Clearly | Clearly | Insoluble |
| preparation | dissolved | dissolved | dissolved | dissolved | matters |
| | | | | | appeared |
| Size of colonies | Average | Average | Large | Large | Large |
| Coloring of colonies | Somewhat | Somewhat | Clear | Clear | Clear |
| | lighter | lighter | | | |
| | than | than | | | |
| | general | general | | | |
| | agar | agar | | | |
| | medium | medium | | | |

As shown in Table 3, both a size and color formation of colonies were satisfactory in the range of 90:10 to 50:50 in a weight ratio of polyvinyl alcohol to methylcellulose. Moreover, solubility during preparation of the culture media was also satisfactory in the range of 90:10 to 75:25.

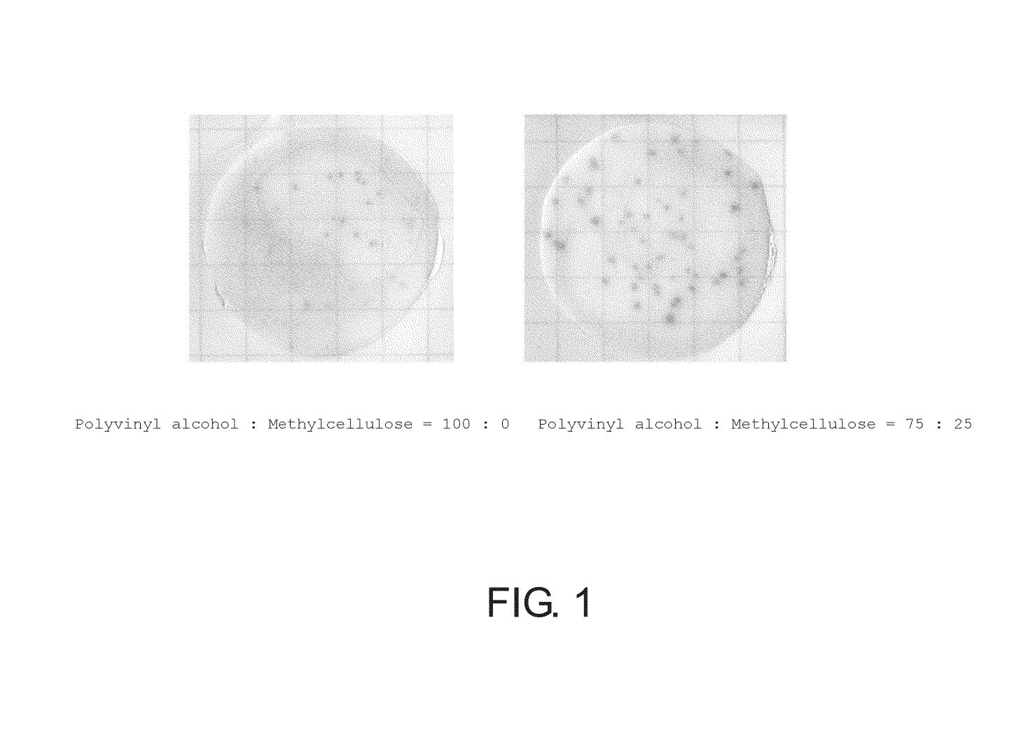

Moreover, FIG. 1 shows photographs of colored colonies on culture media corresponding to simple culture media (1-1) and (1-4). In culture medium (1-4), the colonies were larger and the color formation was stronger and clearer in comparison with culture medium (1-1).

### Example 2

A base culture medium for Staphylococcus aureus in a manner similar to Reference Example, and 1 liter of a gelling agent in a use amount as described in Table 4 were added to 1 liter of purified water, and the resulting mixture was warmed at 95° C. for 1 minute and dissolved with each other. Then, a total amount of the resulting solution was applied onto a polyester film having a thickness of 20 micrometers and 1 m×1 m, and resulting film was dried at 65° C. A product peeled from the polyester film was laminated onto a nylon meltblown nonwoven fabric (90 g/m²). Then, polyester films each having a thickness of 100 micrometers and 70 mm×80 mm were adhered onto a side opposite to the nonwoven fabric, and taken as sheet-form simple culture media (2-1) to (2-7).

In a manner similar to Reference Example, a 10⁻⁷ bacterial diluent of the bacteria solution of Staphylococcus aureus corresponding to McFarland nephelometry No. 1 was applied onto each simple culture medium for testing in an amount of 500 mL/m² for each. The resulting simple culture media were cultured at 35° C. for 24 hours, and then a size and color formation of colonies were confirmed.

TABLE 4

| No. | 2-1 | 2-2 | 2-3 | 2-4 | 2-5 | 2-6 | 2-7 |
|---|---|---|---|---|---|---|---|
| Polyvinyl | 120 | 90 | 90 | 90 | 90 | 90 | 90 |
| alcohol | | | | | | | |
| (g/L) | | | | | | | |
| Xanthan | 0 | 5 | 20 | 25 | 0 | 0 | 0 |
| gum (g/L) | | | | | | | |
| Guar gum | 0 | 0 | 0 | 0 | 5 | 20 | 25 |
| (g/L) | | | | | | | |
| Polyvinyl | 100:0 | 95:5 | 90:20 | 90:20 | 90:5 | 90:20 | 90:25 |
| alcohol: | | | | | | | |
| xanthan | | | | | | | |
| gum or | | | | | | | |
| guar gum | | | | | | | |
| (weight | | | | | | | |
| ratio) | | | | | | | |
| Aspect | Clearly | No mixing | No mixing | No mixing | Clearly | Clearly | Insoluble |
| during | dissolved | | | | dissolved | dissolved | matters |
| preparation | | | | | | | appeared |
| Size of | Average | — | — | — | Large | Large | Large |
| colonies | | (due to no | (due to no | (due to no | | | |
| | | preparation | preparation | preparation | | | |
| | | of culture | of culture | of culture | | | |
| | | medium) | medium) | medium) | | | |
| Color | Somewhat | — | — | — | Clear | Clear | Clear |
| formation | lighter | (due to no | (due to no | (due to no | | | |
| of colonies | than | preparation | preparation | preparation | | | |
| | general | of culture | of culture | of culture | | | |
| | agar | medium) | medium) | medium) | | | |
| | medium | | | | | | |

As shown in Table 4, when polyvinyl alcohol and xanthan gum were combined, no mixing was allowed and no preparation of the culture media was allowed. On the other hand, both a size and color formation of colonies were satisfactory in the range of 90:5 to 90:25 in terms of a weight ratio of polyvinyl alcohol to guar gum. Moreover, solubility during preparation of the culture media was also satisfactory in the range of 90:5 to 90:20.

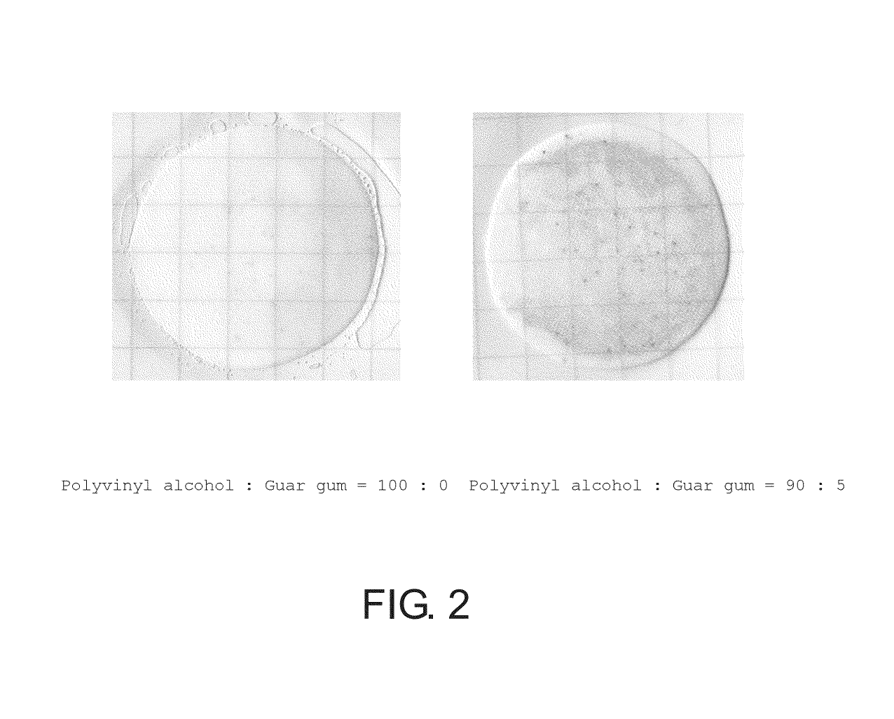

Moreover, FIG. 2 shows photographs of colored colonies on culture media corresponding to simple culture media (2-1) and (2-5). In culture medium (2-5), the colonies were larger, and color formation was stronger and clearer in comparison with culture medium (2-1).

### Example 3

A base culture medium for Coliform bacteria was prepared at a formation shown in Table 5. The base culture medium, and 120 g/L of polyvinyl alcohol or a combination of 90 g/L of polyvinyl alcohol and 5 g/L of guar gum were added to 1 liter of purified water, and the resulting mixture was warmed at 95° C. for 1 minute and dissolved with each other. Then, the resulting solution was applied onto a polyester film having a thickness of 20 micrometers to be 120 g/m² in a total amount of the gelling agent, and the resulting film was dried at 65° C. A product peeled from the polyester film was laminated onto a nylon meltblown nonwoven fabric (90 g/m²). Then, polyester films each having a thickness of 100 micrometers and 70 mm×80 mm were adhered onto a side opposite to the nonwoven fabric, and taken as sheet-form simple culture media (3-1) and (3-2).

TABLE 5
Table 5
Formations of base medium for Coliform (g/m²)

| | |
|---|---|
| Peptone | 1 |
| Meat extract | 4 |
| Bile salt | 2.15 |
| Dipotassium hydrogenphosphate | 0.25 |
| X-GAL | 0.2 |
| Sodium carbonate | 0.05 |

pH 7.4 ± 0.2

Eschericia coli (NBRC 3972) was cultured for 24 hours on a Tryptic Soy Agar (made by Nissui Pharmaceutical Co., Ltd.), and then the resulting bacteria were suspended into sterile physiological saline using a sterile cotton swab so as to correspond to McFarland nephelometry No. 1 (approximately 3.0×10⁸ CFU/mL), and taken as a bacteria stock solution. Then, the bacteria stock solution was repeatedly subjected to 10-fold serial dilution to 10⁻⁷ by using sterile physiological saline, and then a 10⁻⁷ bacterial diluent was applied to each simple culture medium for testing in an amount of 500 mL/m². The resulting culture media were cultured at 35° C. for 24 hours, and then a size and color formation of colonies were confirmed.

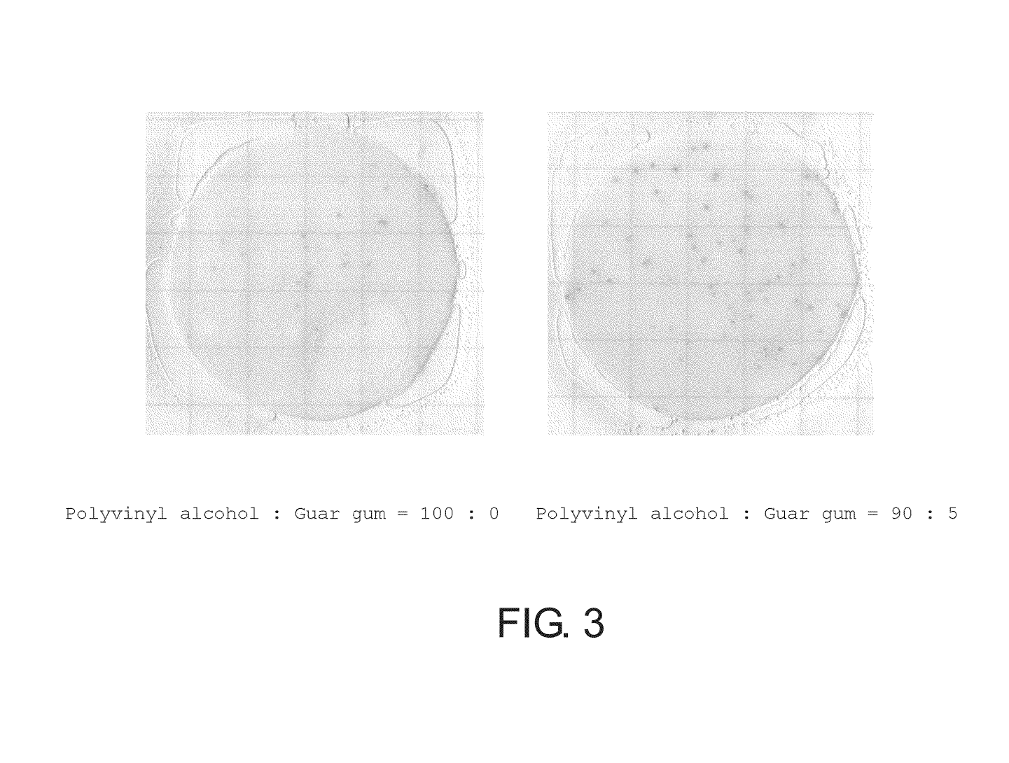

As shown in FIG. 3, the colonies were larger, and color formation of colonies was stronger and clearer in the case of 90:5, in comparison with the case where a weight ratio of polyvinyl alcohol to guar gum was 100:0.

Although the invention has been described and illustrated with a certain degree of particularity, it is understood that the disclosure has been made only by way of example, and that numerous changes in the conditions and order of steps can be resorted to by those skilled in the art without departing from the spirit and scope of the invention.

### INDUSTRIAL APPLICABILITY

The invention provides an inexpensive microorganism culturing material that can simply and efficiently detect microorganisms in a specimen as clear colored colonies, and therefore is industrially significantly useful.

What is claimed is:

1. A microorganism culturing material, comprising a first layer containing a porous material, and a second layer adjacent to the first layer and containing a gelling agent, wherein the gelling agent contains polyvinyl alcohol, and methylcellulose or guar gum.

2. The microorganism culturing material according to claim 1, wherein a weight ratio of polyvinyl alcohol to methylcellulose is 90:10 to 50:50.

3. The microorganism culturing material according to claim 1, wherein a weight ratio of polyvinyl alcohol to guar gum is 90:5 to 90:25.

4. The microorganism culturing material according to claim 1, wherein a total amount of the gelling agent contained in the second layer is 50 to 200 g/m².

5. The microorganism culturing material according to claim 1, wherein the polyvinyl alcohol has a weight average molecular weight of 5,000 to 200,000 and a degree of saponification of 75 to 99%.

6. The microorganism culturing material according to claim 1, further comprising a third layer adjacent to the second layer on a side opposite to the first layer.

7. A microorganism culture medium, comprising the microorganism culturing material according to claim 1, at least one kind of color former or fluorescence agent, and at least one kind of nutritional ingredient.

8. A method for detecting microorganisms, comprising a step of inoculating a specimen to the microorganism culture medium according to claim 7, a step of culturing microorganisms contained in the specimen, and a step of detecting colonies of the microorganisms.
